(19) United States
(12) Patent Application Publication
(10) Pub. No.: US 2026/0183324 A1
(43) Pub. Date: Jul. 2, 2026

(54) FORMULATIONS FOR BOOSTING NAD+ USING MULTIPLE BIOLOGICAL PATHWAYS CONCURRENTLY TO OVERCOME ENZYMATIC DEGRADATION AND IMPROVE DOWNSTREAM BENEFITS

(71) Applicants: Nicholas Bitz, Irvine, CA (US); Angelica S Vrablic, Delray Beach, FL (US); Daniel C Obegi, El Segundo, CA (US)

(72) Inventors: Nicholas Bitz, Irvine, CA (US); Angelica S Vrablic, Delray Beach, FL (US); Daniel C Obegi, El Segundo, CA (US)

(73) Assignee: PROHEALTH, Carpinteria, CA (US)

(21) Appl. No.: 19/428,785

(22) Filed: Dec. 22, 2025

Related U.S. Application Data
(60) Provisional application No. 63/740,245, filed on Dec. 30, 2024.

(51) Int. Cl. A61K 31/706; A61K 9/00; A61K 31/352; A61K 31/405; A61K 31/455

(52) U.S. Cl. CPC A61K 31/706; A61K 9/0053; A61K 31/352; A61K 31/405; A61K 31/455

(57) ABSTRACT

Orally-ingestible supplement formulations for boosting NAD+ (Nicotinamide adenine dinucleotide) in a human and methods of use. The formulations provide a Triple Boost for NAD+ that will combine 3 or more different ingredients each of which uses a different metabolic pathway selected from the following: a) the De Novo Pathway, b) the Preiss-Handler Pathway, c) the Salvage Pathway, d) the Reduced NAD+ Precursor Pathway, and e) CD38 Inhibition Pathway.

### RELATED APPLICATIONS

The present application claims priority to U.S. provisional Ser. No. 63/740,245, filed Dec. 30, 2024.

### FIELD OF THE INVENTION

The present application relates generally to orally-ingestible formulations and methods of use for boosting NAD+ (Nicotinamide adenine dinucleotide) in a human.

### BACKGROUND OF THE INVENTION

NAD+ (Nicotinamide adenine dinucleotide, often just “NAD”) is a coenzyme found in every mammalian cell that's critical for over 200 biological processes. NAD+ is so important that death follows instantly without NAD+. NAD+ declines as we age at a rate of about 1% per year and as it declines cell function declines which leads to aging and disease.

Despite a large number of supplements and other compositions intended to boost the presence of NAD+ in humans, there remains a need for a more efficient formulation because current formulations are rate limited, and do not account for degradation/consumption pathways which creates an upper limit for efficacy.

### SUMMARY OF THE INVENTION

The present application describes orally-ingestible supplement or drug formulations for boosting NAD+ (Nicotinamide adenine dinucleotide) in a human. The formulations provide a so-called Triple Boost for NAD+ by combining 3 or more different ingredients that use at least 3 distinct metabolic pathways selected from the group consisting of the following:
1. De Novo Pathway
2. Preiss-Handler Pathway
3. Salvage Pathway
4. Reduced NAD+ Precursor Pathway
5. CD38 Inhibition

The present application discloses a composition comprising NAD+ precursors and CD38 inhibitors, wherein the ingredients work complementary to:
a. Counter the effects of aging.
b. Boost NAD+synthesis via multiple pathways.
c. Inhibit enzymatic degradation of NAD+.

A method is disclosed for increasing NAD+ levels and activity in a human by administering a therapeutically effective dose of the composition described, resulting in improved cellular function and health outcomes.

A method for boosting NAD+ in a human in need thereof by orally self-administering a therapeutically effective amount of a formulation comprising at least 3 distinct metabolic pathways selected from the group consisting of the following:
a. De Novo Pathway,
b. Preiss-Handler Pathway
c. Salvage Pathway,
d. Reduced NAD+ Precursor Pathway, and
e. and CD38 Inhibition.

The present application provides a Triple Boost formula for NAD+ by combining 3 or more different ingredients that use at least 3 distinct metabolic pathways as described herein. In some instance, at least 4 pathways are used, and in some cases at least 5 pathways. Five distinct pathways are disclosed, and thus the application contemplates at least 3 of 5, at least 4 of 5, and all 5 of the 5 pathways. If the pathways are given the shorthand a-b-c-d-e as disclosed in the previous paragraph, the possible combinations are abc, abd, abe, acd, ace, ade, bcd, bce, cde, bde, abcd, abce, abde, acde, bcde and abcde.

### BRIEF DESCRIPTION OF THE DRAWINGS

Features and advantages of the present invention will become appreciated as the same become better understood with reference to the specification, claims, and appended drawings wherein:

FIG. 1 is a Table showing an exemplary formulation of an orally-ingestible Triple Boost composition designed to boost NAD+ in a human.

### DETAILED DESCRIPTION OF THE PREFERRED EMBODIMENTS

The present invention provides a number of preferred formulations of supplements for boosting NAD+ (Nicotinamide adenine dinucleotide) in a human.

The present application provides orally-ingestible compositions to provide a Triple Boost for NAD+ production that will provide a) a source of NAD+ or NAD+ precursors that are converted in the body to NAD+ and b) inhibitors to keep NAD+ from being metabolized.

For example: Niacin is a dietary precursor that is metabolized to NAD+, and apigenin protects NAD+ from being metabolized and also activates sirtuins. B vitamins and TMG can assist with methylation.

Other examples: Tryptophan, Niacinamide, NR (Nicotinamide riboside), NMN (Nicotinamide mononucleotide), and NADH are all precursors that can lead to increased NAD+ levels.

NAD+ can be boosted with supplements but no single supplement can boost NAD+ levels enough to achieve the optimal cellular function of youthful cells in adults. The NAD+ increase from supplements is limited though by the time it takes to metabolize the supplement ingredients. Our bodies can only absorb a finite amount of a nutrient per hour, and enzymatic conversion/activation is rate-limited. The formulations described herein are designed to maximize the uptake, conversion and retention of NAD+, more so than any other single pathway supplement can.

There are multiple biological pathways to increase NAD+. To help illustrate the point, a good metaphor is a freeway. On a freeway there's an upper limit to how many cars can pass each hour in each lane no matter what the qualities of the freeway and car are, but by adding lanes the volume of cars per hour increases significantly. Like how a freeway with four lanes allows four times the number of cars to pass per hour, using four biological pathways concurrently to boost NAD+ increases the total NAD+ boosting potential. By combining supplement ingredients that use multiple different pathways we're able to improve health by boosting NAD+ significantly more than what's possible by using any one pathway. Since so many diseases are caused by low NAD+ this invention has the potential to improve the lives of, and even save the lives of millions of people per year.

The five potential pathways are:
1. V—De Novo Pathway—The De Novo pathway for NAD+ synthesis is a biochemical process where NAD+ is produced from tryptophan through a series of enzymatic reactions, converting it into quinolinic acid and then into NaMN (nicotinic acid mononucleotide) before forming NAD+. The De Novo pathway converts tryptophan into NAD+ through the kynurenine route. Example steps include:
   Tryptophan is converted to kynurenine.
   Kynurenine is converted to quinolinic acid.
   Quinolinic acid is converted to NAMN by QPRT.
   NAMN proceeds through the same steps as the Preiss Handler Pathway to produce NAD+.
2. W—Preiss-Handler Pathway—The Preiss-Handler pathway synthesizes NAD+ by converting nicotinic acid (NA) into NAD+. More specifically, Preiss-Handler pathway converts NA (nicotinic acid) into NaMN (nicotinic acid mononucleotide) and then into NaAD (nicotinic acid adenine dinucleotide), which is finally amidated to form NAD+. For instance, the Preiss Handler Pathway is a process that converts Vitamin B3 (i.e., Niacin) into NAMN. Example steps include:
   NA is converted to NAMN by NAPRT.
   NAMN is converted to NAAD by NMNAT.
   NAAD is converted to NAD+ by NADS.
3. X—Salvage Pathway (involving second-order NAD boosters)—The Salvage pathway for NAD+ recycles nicotinamide (NAM) or NA (nicotinic acid) into NAD+ through a series of enzymatic steps involving their conversion to intermediates like NMN (Nicotinamide mononucleotide) or NaMN (nicotinic acid mononucleotide), bypassing de novo synthesis. NR (Nicotinamide riboside) bypasses NAMPT (Nicotinamide phosphoribosyltransferase) and is intracellulary metabolized by kinases NRK1 and NRK2 to NMN. NMN is transformed to NAD+ by NMNATi-3 (Nicotinamide mononucleotide adenyltransferases). Finally, the vitamin B3 analog Niacinamide is now believed to bypass the rate-limiting enzyme NAMPT of the Priess-Handler pathway. All of these are grouped herein as second-order NAD boosters. Example steps include:
   NAM is converted to NMN by NAMPT.
   NMN is converted to NAD+ by NMNAT.
4. Y—Reduced NAD+ Precursor or NMNH Pathway uses reduced nicotinamide mononucleotide to produce NADH. For instance, NRH (Dihydronicotinamide riboside) and Reduced nicotinamide mononucleotide (NMNH, also known as reduced NMN or Beta Dihydronicotinamide mononucleotide) are reduced NAD+ precursors that are converted to NADH (Nicotinamide Adenine Dinucleotide+H) by NMNAT (NMN adenyltransferases), increasing cellular NADH levels, which can then be oxidized to replenish NAD+. Example steps include:
   NADH is oxidized to NAD+.
   NADH is used to produce ATP.
5. Z. CD38 Inhibition—The suppression of CD38, an enzyme that breaks down NAD+ into ADP-ribose and nicotinamide, helps conserve NAD+ levels.

Example ingredients that each use a distinct metabolic pathway are:
1. De Novo Pathway: L-tryptophan
2. Preiss-Handler Pathway: Nicotinic acid (niacin), trigonelline
3. Salvage Pathway: Niacinamide, NR, NMN
4. Reduced NAD+ Precursors: NMNH, NRH
5. CD38 Inhibition: Apigenin, quercetin, fisetin, pterostilbene, resveratrol, curcumin and spermidine.

Example dose ranges of ingredients for each pathway are:
1. A: Tryptophan 60 mg
2. B: Niacin 20 mg, Trigonelline 20 mg
3. C: Niacinamide 150 mg, NR 500 mg, NMN 500 mg
4. D: NRH 125 mg NMNH 125 mg
5. E. Apigenin 50 mg, Quercetin 50 mg

Where A=(10-120 mg), B=(5-60 mg), C=(50-500 mg), D=(100-500 mg), and E=(10-200 mg), and where these ranges apply to either or both of the ingredients in each pathway.

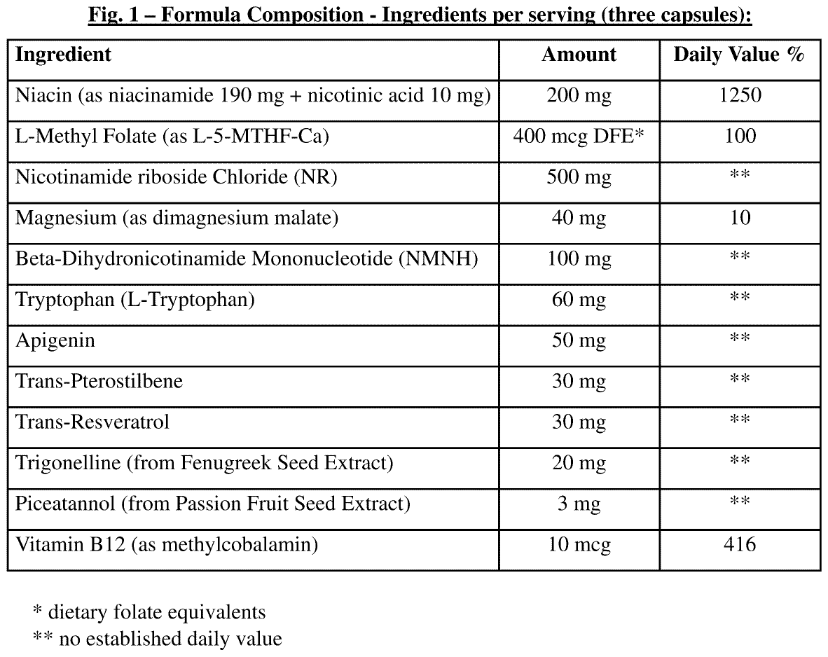

FIG. 1 is a Table showing an exemplary formulation of an orally-ingestible Triple Boost composition designed to boost NAD+ in a human, and percentage of suggested daily value for each. A formulation as in FIG. 1 may be orally ingested at a dose of 1300 mg per day (once) for a period of one month (or longer if needed), and a boost in NAD+ production and retention is found to be 50% to 100% higher than baseline amount based on human studies (observational and NAD+ booster interventions). For example, 1000 mg/day of NR increased NAD+ blood concentrations two-fold in elderly subjects (Elhassan Y S, et al. Cell Rep. 2019 August 13; 28(7):1717-1728.e6. doi: 10.1016/j.celrep.2019.07.043. PMID: 31412242; PMCID: PMC6702140. We should expect our formula to increase NAD+ levels in the blood by at least two-fold without the need for high doses (1000 mg/day) of an NR booster. Note: no studies have been done on a Triple Boost formula so the increase would be the sum of the observed increases for single pathway interventions that have been tested.

As a matter of foundational support, the B vitamin niacin (B3) is part of a family of compounds that are essential for energy metabolism, cell growth and differentiation among other important functions. This “niacin family” of compounds includes (among others) nicotinamide (nicotinic acid amide) CAS #98-92-0; nicotinic acid CAS #59-67-6, NMN (Nicotinamide mononucleotide) CAS #1094-61-7; and NR (Nicotinamide riboside) CAS #2311-00-4; as well as the amino acid tryptophan which can be converted to nicotinamide. (Institute of Medicine. 2000. Dietary Reference Intakes: For Thiamin, Riboflavin, Niacin, Vitamin B₆, Folate, Vitamin B₁₂, Pantothenic Acid, Biotin and Choline. Washington, D.C.: The National Academies Press.)

Overall, the most important function is the production of nicotinamide adenine dinucleotide, NAD+, a coenzyme (molecule that binds with proteins) present in all living cells. NAD+ acts as a hydride ion acceptor or donor in many biological redox reactions. The most well-known function of NAD+ is the transferring of electrons to produce ATP. (Institute of Medicine. 2000. Dietary Reference Intakes.)

NAD+ also activates sirtuins, enzymes associated with a wide variety of functions related to metabolism and longevity, and PARPs enzymes associated with DNA repair and cell growth/function. (Institute of Medicine. 2000. Dietary Reference Intakes.)

Production of NAD+: NAD+ production involves several key pathways. The De Novo pathway synthesizes NAD+ from tryptophan, while the Preiss-Handler pathway converts nicotinic acid (niacin) or trigonelline into NAD+. The Salvage pathway recycles precursors like nicotinamide, NR, and NMN to regenerate NAD+, and the reduced precursors pathway uses NRH and NMNH to boost NADH levels, which can be oxidized to replenish NAD+. Additionally, CD38 inhibition helps preserve NAD+ levels by reducing its enzymatic degradation. (doi: 10.1096/fj.202001826, doi: 10.1186/s12929-019-0527-8, doi: 10.1016/j.molmed.2017.08.001, doi: 10.1016/j.cmet.2016.05.006)

NAD+ is consumed and degraded to generate nicotinamide and ADP-ribose by several enzymes such as PARPS, and NAD+ase (NAD+ glycohydrolase) such as CD38. CD38 is the main cellular NAD+ hydrolase in mammalian tissues and regulates cellular levels of NAD+. (Escande C, Nin V, Price N L, Capellini V, Gomes A P, Barbosa M T, O'Neil L, White T A, Sinclair D A, Chini E N. Flavonoid apigenin is an inhibitor of the NAD+ase (NAD+ glycohydrolase) such as CD38: implications for cellular NAD+ metabolism, protein acetylation, and treatment of metabolic syndrome. Diabetes. 2013 April; 62(4):1084-93. doi: 10.2337/db12-1139. Epub 2012 Nov. 19. PMID: 23172919; PMCID: PMC3609577.)

CD38 is inhibited by flavonoids such as quercetin and apigenin. (Escande, C. et al. Diabetes. 2013)

Increasing NAD+ levels: B3 analogues such as niacinamide and nicotinic acid are first-order NAD+ precursors that increase NAD+ levels, while NR and NMN are likely more efficient because they can bypass the rate-limiting enzyme (NAMPT) of the Preiss-Handler pathway. However, NAD+ can also be consumed by NAD+ hydrolases such as CD38, so inhibition of the CD38 enzyme will sustain the increase and availability of NAD+ by decreasing consumption and degradation.

Suggested Boosters: A Triple Boost formula for NAD+ production will provide NAD+ precursors to increase natural production, and inhibitors of CD38 to keep NAD+ from being metabolized.

Niacin is a dietary source that is metabolized to NAD+, as is the amino acid tryptophan. NR (Nicotinamide riboside) is a source of NAD+, and flavonoids such as quercetin and apigenin protect NAD+ from being metabolized by NADases such as CD38. (Escande, C. et al. Diabetes. 2013)

Quercetin, resveratrol and pterostilbene also activate sirtuins. Additionally, B vitamins (B2, B6 and B12) provide coenzyme support for nucleotide metabolism and methylation. (Institute of Medicine. 2000. Dietary Reference Intakes.)

While the invention has been described in its preferred embodiments, it is to be understood that the words that have been used are words of description and not of limitation. Therefore, changes may be made within the appended claims without departing from the true scope of the invention.

What is claimed is:

1. An orally-ingestible formulation for boosting NAD+ in a human by combining 3 or more different ingredients each of which uses a different metabolic pathway selected from the group consisting of the following:
a) a De Novo Pathway,
b) a Preiss-Handler Pathway,
c) a Salvage Pathway or second-order NAD boosters,
d) a Reduced NAD+ Precursor Pathway, and
e) a CD38 Inhibition Pathway.

2. The formulation of claim 1, wherein the ingredient that uses the De Novo Pathway is L-tryptophan.

3. The formulation of claim 2, wherein one serving has 10-120 mg of L-tryptophan.

4. The formulation of claim 1, wherein the ingredient that uses the Preiss-Handler Pathway is selected from the group consisting of Nicotinic acid (niacin) and trigonelline.

5. The formulation of claim 4, wherein one serving has 5-60 mg of Nicotinic acid (niacin) and 5-60 mg of trigonelline.

6. The formulation of claim 1, wherein the ingredient that uses the Salvage Pathway is selected from the group consisting of Niacinamide, NMN (Nicotinamide mononucleotide) and NR (Nicotinamide riboside).

7. The formulation of claim 6, wherein one serving has 50-500 mg Niacinamide and 50-500 mg NR (Nicotinamide riboside).

8. The formulation of claim 1, wherein the ingredient that uses the Reduced NAD+ Precursor Pathway is Beta Dihydronicotinamide mononucleotide (NMNH).

9. The formulation of claim 8, wherein one serving has 100-500 mg of Beta Dihydronicotinamide mononucleotide (NMNH).

10. The formulation of claim 1, wherein the ingredient that uses the CD38 Inhibition Pathway is selected from the group consisting of apigenin and quercetin.

11. The formulation of claim 10, wherein one serving has 10-200 mg of either apigenin or quercetin.

12. The formulation of claim 1, wherein one of the at least 3 distinct metabolic pathways is the CD38 Inhibition Pathway.

13. A method for boosting NAD+ in a human in need thereof by orally self-administering a therapeutically effective amount of the formulation of claim 1.

14. The method of claim 13, wherein the ingredient that uses the De Novo Pathway is L-tryptophan.

15. The method of claim 14, wherein one serving has 10-120 mg of L-tryptophan.

16. The method of claim 13, wherein the ingredient that uses the Preiss-Handler Pathway is selected from the group consisting of Nicotinic acid (niacin) and trigonelline.

17. The method of claim 13, wherein the ingredient that uses the Salvage Pathway is selected from the group consisting of Niacinamide, NMN (Nicotinamide mononucleotide) and NR (Nicotinamide riboside).

18. The method of claim 13, wherein the ingredient that uses the Reduced NAD+ Precursor Pathway is Beta Dihydronicotinamide mononucleotide (NMNH).

19. The method of claim 13, wherein the ingredient that uses the CD38 Inhibition Pathway is selected from the group consisting of apigenin and quercetin.

20. The method of claim 13, wherein one of the at least 3 distinct metabolic pathways is the CD38 Inhibition Pathway.
